(19) United States
(12) Patent Application Publication
(10) Pub. No.: US 2006/0189849 A1
(43) Pub. Date: Aug. 24, 2006

(54) Surgical illumination device and method of using

(76) Inventors: Todd W. Sharratt, Stillwater, MN (US); Ronald S. Von Wald, Centerville, MN (US)

Correspondence Address: Richard C. Emery;Attorney at Law, 4189 Lakewood Avenue, White Bear Lake, MN, 55110-3925, US

(21) Appl. No.: 11/248,810

(22) Filed: Oct. 12, 2005

Related U.S. Application Data
(60) Provisional application No. 60/655,153, filed on Feb. 22, 2005.

(51) Int. Cl. A61B 1/06

(52) U.S. Cl. 600/249

(57) ABSTRACT

A surgical illumination device and method of using includes a mounting platform attachable to a surgical device and light conducting fibers attached to the platform.

This Application claims the benefit of Provisional Patent Application 60/655,153 filed Feb. 22, 2005 and is herein incorporated by reference in its entirety.

### FIELD OF THE INVENTION

The invention relates to a surgical illumination device for lighting an exposed area during medical or surgical treatment. The device is separately attachable to a variety of surgical or medical devices.

### BACKGROUND

Medical treatment often requires a surgeon to create an incision in a patient to access the anatomical site requiring treatment. Any incision results in some amount of trauma to the patient, including pain, potential infection, healing time and dysfunction. A smaller incision generally reduces trauma and is thus a goal of all surgical treatment. In order to most effectively access the interior of a patient, retractor systems have been developed which allow a physician sufficient anatomical access while minimizing patient trauma through a smaller incision. A system developed and sold by Omni-Tract Surgical, Inc., a division of Minnesota Scientific, Inc., St. Paul, Minn. achieves this goal by providing a surgical retractor system which is mounted to an operating table and inserted into a minimal incision and then able to be firmly locked into any configuration required by the physician throughout the duration of the procedure (see U.S. Pat. Nos. 5,400,772, 5,727,899, 5,899,627 and 6,042,541, all of which are hereby incorporated by reference). Thus, a minimal incision is created which still allows the physician ample room to be able to perform the procedure. A problem that exists with operating through a minimal incision, however, is providing sufficient illumination to enable the physician to see what he/she is doing. In traditional surgical technique, a larger incision allowed the use of distantly mounted, relatively powerful lights to illuminate the exposed interior of the patient's body. The recent advent of minimal sized incisions, however, creates the need for another way to illuminate the treatment site.

Illuminated surgical retractors are known in the art. Lumitex®, Inc., Strongsville, Ohio has developed an illuminated panel made of sandwiched, woven optical fibers that can be attached to the flat blade of a traditional surgical retractor by adhesive wings. The panel is connected to a high intensity light source via an optical fiber and provides light to the interior of an exposed surgical treatment site. Because a woven optical fiber panel lighting system requires hand labor to manufacture, such a lighting system is expensive to manufacture. The Lumitex® retractor, as well as other lighted retractors, are also specific to certain retractor designs and not readily usable on varying sizes and retractor types. Further, these retractors take up a great deal of space in the wound, thus requiring larger incisions and making the surgeon's job more difficult. Finally, these light types are not readily moved from retractor to retractor after initial placement. What is clearly needed, therefore, is a surgical illumination device that is inexpensive and easily adaptable to a wide variety of surgical and medical instruments.

### SUMMARY

In one aspect, the invention comprises a device for securing light conducting fibers to a surgical retractor blade. The device includes a mounting platform attachable to a surgical device and means for attaching least one light conducting fiber to the mounting platform.

In another aspect, the platform is a clip defining a first end and a second end, the first and second ends almost meeting each other to form a gap which when moved apart exert a force toward each other.

In yet another aspect, the platform is a pad defining a first major surface and a second major surface.

In an alternative aspect, the invention comprises a surgical illumination device, including amounting platform attachable to a surgical device and at least one light conducting fiber attached to the platform.

In a further aspect, the platform is a clip defining a first end and a second end, the first and second ends almost meeting each other to form a gap which when moved apart exert an amount of force toward each other.

In another aspect, the platform is a pad defining a first major surface and a second major surface.

In still another aspect, the invention comprises an illumination device having a mounting platform attachable to a surgical device. At least one light conducting fiber is attached to the mounting platform and a light source is in light communication with the at least one light conducting fiber to conduct light energy through the at least one light conducting fiber.

In an additional aspect, the invention comprises a method of using a surgical illumination device, comprising the steps of: (a) providing a mounting platform attachable to a surgical device, the platform having at least one light conducting fiber attached to it; (b) attaching the mounting platform to a surgical device; (c) connecting the at least one light conducting fiber to a light source; (d) locating the surgical device, attached mounting platform and at least one light conducting fiber at a patient's medical treatment site; and (e) energizing the light source, thereby illuminating the medical treatment site.

### BRIEF DESCRIPTION OF THE DRAWINGS

FIG. 1 shows a plan view of an embodiment of the invention having light conducting fibers attached to a clip which attaches to a surgical retractor blade.

FIG. 2 shows a cross-section of the light conducting fiber bundle taken between the points 2-2 as shown in FIG. 1.

FIG. 3 shows a perspective view of a clip in a stressed configuration with light conducting fibers attached, which is attached to a retractor blade.

FIG. 3A shows an end view of the clip shown in FIG. 3 in the unstressed configuration, without light conducting fibers attached.

FIG. 3B shows an end view of the clip shown in FIG. 3 in the unstressed configuration, with light conducting fibers attached.

FIG. 4 shows a perspective view of an alternative embodiment of the invention having light conducting fibers attached to a pad having an adhesive surface for attachment to a surgical retractor blade.

FIG. 4A shows an end view of an embodiment of a pad having the light conducting fibers directly attached to a first major surface of the pad by means of an adhesive.

FIG. 4B shows an end view of the pad shown in FIG. 4 having an adhesive attachment layer with light conducting fibers attached to the retaining channels.

FIG. 5A shows an end view of an embodiment of a clip in the unstressed configuration, without any light conducting fibers attached.

FIG. 5B shows the clip shown in FIG. 5A in the unstressed configuration with light conducting fibers attached to the first major surface of the clip by means of an adhesive.

FIG. 6 shows a clip having light conducting fibers attached, which is attached to the blade of a surgical retractor during a surgical procedure.

### DETAILED DESCRIPTION

Definitions

“Pull Force” refers to an amount of force required to remove a first object from a second object to which the first object is attached.

### NOMENCLATURE

200 Surgical Illumination Device
202 Light Source
203 Connector
204 Light Conducting Fiber Bundle
205 Light Conducting Fiber
206 Retractor Blade
207 Sheath
208 Clip
209 First End of Clip
210 Retaining Channel
211 Second End of Clip
213 Gap
215 First Major Surface
300 Surgical Illumination Device
302 Light Source
304 First Major Surface
305 Adhesive
306 Second Major Surface
308 Pad
310 Pressure Sensitive Adhesive
400 Surgical Illumination Device
407 Adhesive
408 Clip
409 First End of Clip
411 Second End of Clip
413 Gap
415 First Major Surface
600 Surgical Illumination Device
602 First Major Surface
604 Second Major Surface
606 Retaining Channel
608 Pad
610 Pressure Sensitive Adhesive
1000 Leg
   Construction

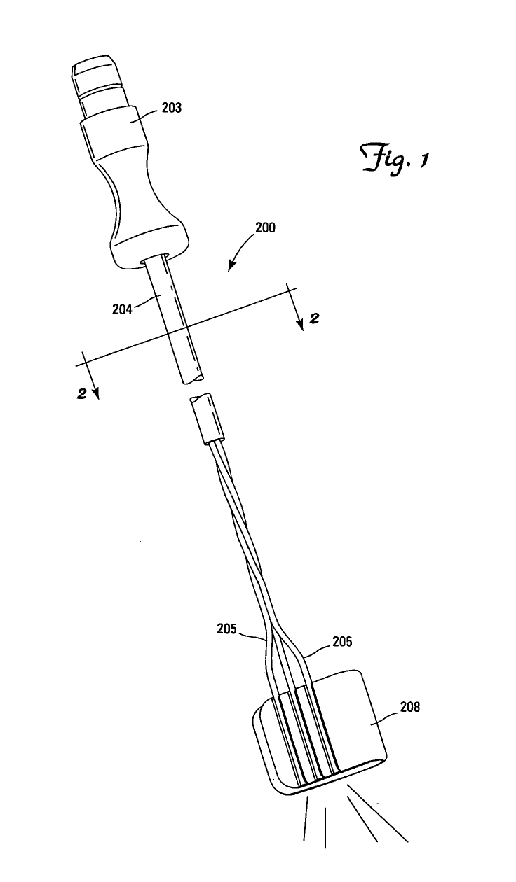

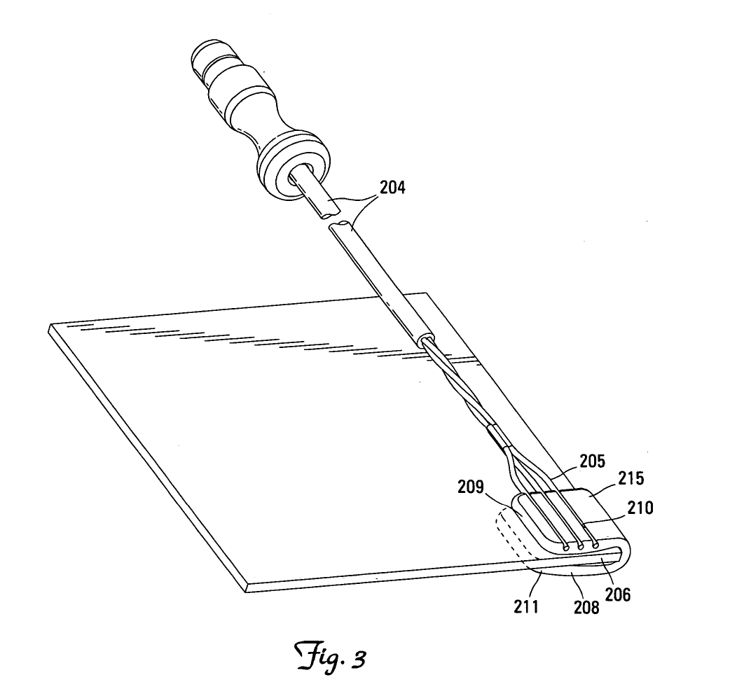

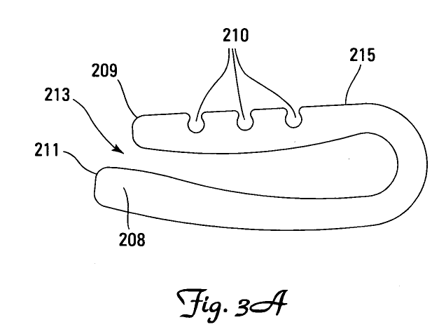

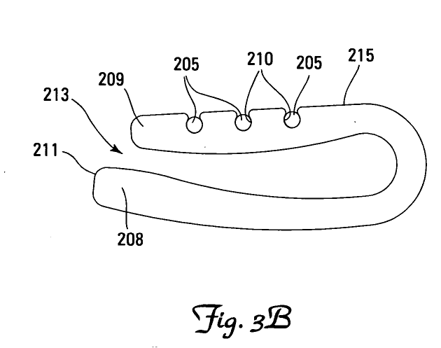

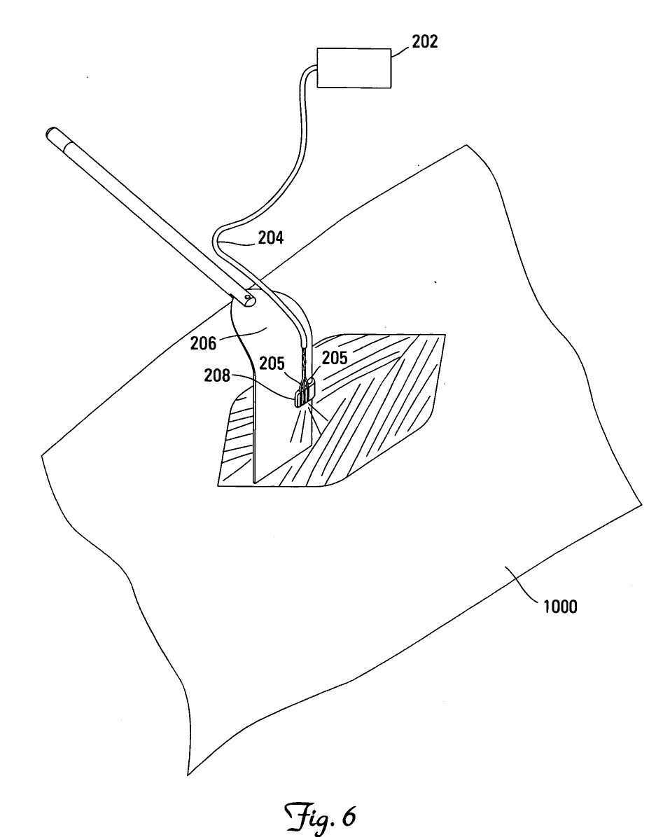

FIG. 1 shows a plan view of an embodiment of the surgical illumination device 200 of the present invention. In this embodiment, the invention comprises a clip 208, best shown in FIGS. 3, 3A, 3B which is formed to fold upon itself so that a first end 209 and second end 211 almost meet to form a gap 213. In another embodiment (not shown), the first end (not shown) and second end (not shown) do meet when the clip (not shown) is not attached to a surgical instrument. Whether or not the first and second ends of the clip 208 meet is not critical to the functioning of the invention. Before the clip 208 is slid over an edge of a surgical retractor blade 206 as shown in FIGS. 3 and 6, or other medical or surgical instrument (not shown), the clip 208 is formed so the gap 213 defines a distance which is less than the width of the retractor blade 206. In the embodiment (not shown) where the first end (not shown) and second end (not shown) actually contact each other, the width of the retractor blade 206 is less important as the gap 213, by definition, defines a lesser distance than the width of the retractor blade 206. In both embodiments, upon installing the clip 208 onto a surgical instrument, the first 209 and second ends 211 of the clip 208 are forced apart, which exerts an amount of force sufficient to securely attach the clip 208 to the retractor blade 206 during a surgical procedure. Most retractor blades 206 have a thickness between 0.048 inches and 0.105 inches. The average pull force required to securely attach the clip 208 to a 0.05 gage retractor blade 206 is approximately 1.67 pounds but may vary according to specific requirements between approximately 1.54 and 1.86 pounds. The average pull force required to securely attach the clip 208 to a 0.105 gage retractor blade 206 is approximately 4.64 pounds but may vary according to specific requirements between approximately 4.40 and 4.60 pounds. The clip 208 and attached light conducting fibers 205 are thus adjustably movable according to the desires of the physician. It is also contemplated by and therefore within the scope of the invention to add a coating (not shown) or textured inner surface (not shown) or altered inner surface geometry (not shown) to the contacting surfaces of the clip (not shown), which increases the holding ability of the clip without increasing the exerted force.

The clip 208 is preferably made of a medically and biologically compatible plastic such as Lustran® ABS plastic, made by Lanxess Engineering Plastics and can be machined, extruded or injection molded. Alternative materials could also be used such as stainless steel or nitinol, however, plastic materials are preferred due to low cost and high performance.

In the embodiment shown in FIGS. 1, 3, 3A, 3B, and 6, the clip 208 defines a first major surface 215 into which at least one retaining channel 210 at least partly extends across the first major surface 215. The retaining channel 210 is preferably a partial circle cut or molded into the first major surface 215 having the majority of the circle below the first major surface 215, thus allowing the light conducting fiber 205 to be popped in and out of the retaining channel as required.

The clip 208 has attached to it at least one and preferably a plurality of light conducting fibers 205 which are attached to and in light communication with a light source 202. Light conducting fibers 205, commonly known as “fiber optics” are made of polymethyl methacrylate. The light source is a well known xenon type such as manufactured by Wolf, CUDA, Karl Stortz, Wehmerlite and Olympus. Light intensity can be adjusted at the light source 202 as required by the physician.

FIG. 3 shows a perspective view of an embodiment of the surgical illumination device 200 of the present invention. In this embodiment a retractor blade 206 is provided which can be attached to a table mounted retractor system (not shown) or may alternatively be hand held by a technician (not shown). As shown in cross section in FIG. 3a, the clip 208 is configured to be quickly attachable to the retractor blade 206 by means of a friction or compression fit. It should be mentioned that it is contemplated to configure the device 200 to be attachable to any medical device, thus the invention is not limited to use with surgical retractors.

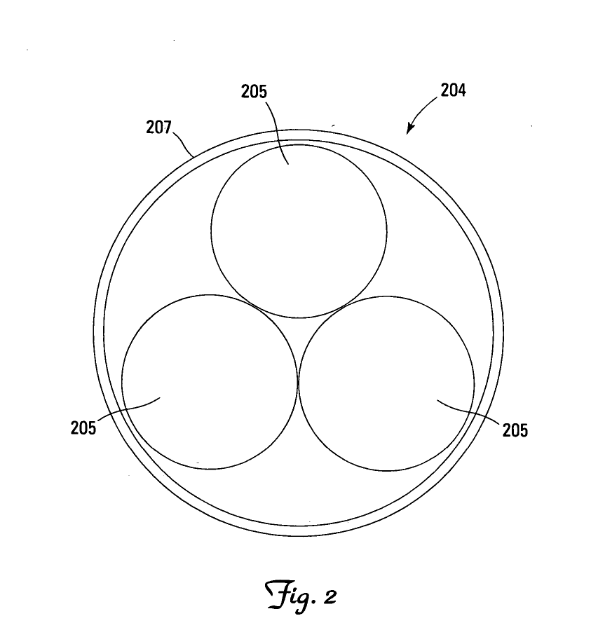

Attached to the clip 208 is at least one light conducting fiber 205 which is in light communication with a light source 202. The light conducting fiber has core of polymethyl methacrylate and is clad with a fluorinated polymer; light conducting fibers 205 are well known in the art and are sold by Moritex, Inc., Tokyo, Japan. As shown in FIGS. 1 and 2, a light conducting fiber bundle 204 may be used instead of a single light conducting fiber 205 and comprises a plurality of light conducting fibers 205 encased in a sheath 207. The sheath 207 is made of a material such as 85 Shore PVC, such as Lynn Part Number LVD-165 made by Lynn Plastics Corp., in a diameter of approximately 0.135 inches when encasing three light conducting fibers 205. Different numbers or diameters of light conducting fibers 205 will, of course, require a different sized sheath 207. A standard snap fit connector 203 terminates the light conducting fiber bundle 204 or single light conducting fiber 205 and is configured to plug into the light source 202. An advantage of using a clip 208 mounted system is that it can quickly and easily be moved on the medical device as individual procedures may require.

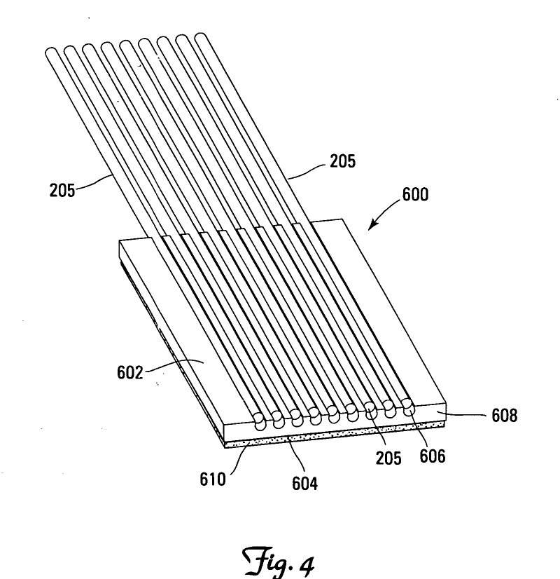

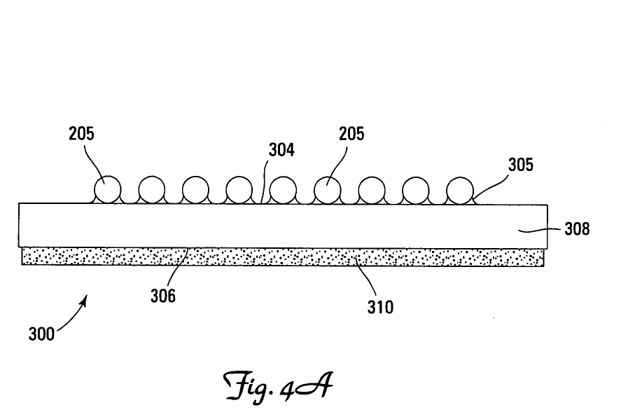

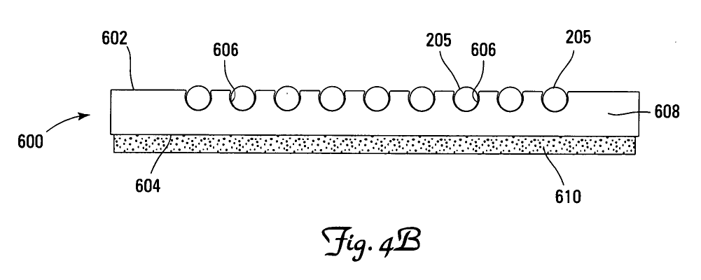

FIGS. 4 and 4B show a perspective view of yet another embodiment of the surgical illumination device 600 of the present invention. As shown in cross section in FIG. 4B, a pad 608 is configured to be quickly attachable to the surgical device by means of a pressure sensitive adhesive 610 affixed to a second major surface 604. Attached to the pad 608 is at least one and preferably a plurality of light conducting fibers 205 which are in light communication with a light source (not shown for this embodiment, but similar to the light source 202 as discussed herein).

The pad 608 is provided with at least one retaining channel 606 which extends at least partly across a first major surface 602. The at least one retaining channel 606 is preferably a partial circle cut or molded into the pad 608, having the majority of the circle below the first major surface 602. The retaining channel 606 opens through the first major surface 602, thus allowing the light conducting fiber 205 to be popped in and out of the retaining channel 606 as required. While a flat pad 608 is shown in FIGS. 4 and 4B, additional embodiments including curved (not shown), compound (not shown), complex (not shown) or having a surface specifically configured to conform to a particular surgical instrument (not shown) are also contemplated by and therefore within the scope of the invention. The pad 608 can be made of a wide variety of plastic, metallic or composite materials as required and machined, extruded or injection molded.

FIG. 4A shows an alternative embodiment of the surgical illumination device 300. A pad 308 is configured to be quickly attachable to a surgical device (not shown) by means of pressure sensitive adhesive 310 attached to the second major surface 306. Attached to the first major surface 304 of the pad 308 is at least one light conducting fiber 205 which is in light communication with a light source (not shown). The light conducting fiber 205 is attached to the pad 308 by means of an adhesive 305 such as Bayer Lustran ABS. Additional methods of attachment such as magnetic (not shown) are also contemplated by and therefore within the scope of the invention.

While a flat pad 608 is shown in FIG. 4A, additional embodiments including curved (not shown), compound (not shown), complex (not shown) or having a surface specifically configured to conform to a particular surgical instrument (not shown) are contemplated by and therefore within the scope of the invention.

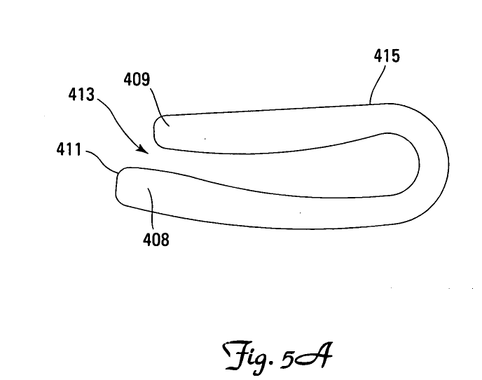

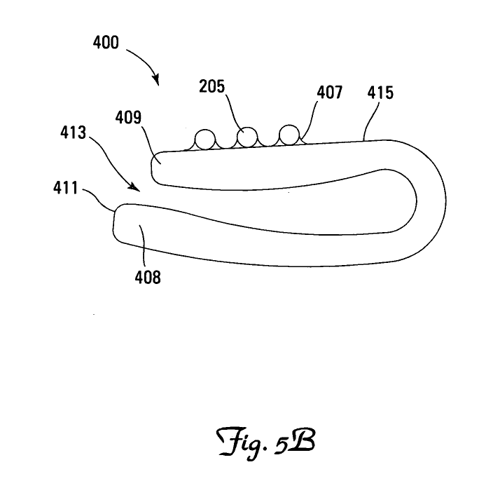

FIGS. 5A and 5B show yet another embodiment of the surgical illumination device 400. In this embodiment, a clip 408 is provided which is similar to the clip 208 shown in FIGS. 1, 3, 3A, 3B and 6. In this embodiment, the invention comprises a clip 408 which is formed to fold upon itself so that a first end 409 and second end 411 almost meet to form a gap 413. In another embodiment (not shown), the first end (not shown) and second end (not shown) do meet when the clip (not shown) is not attached to a surgical instrument. Whether or not the first 409 and second ends 411 of the clip 408 meet is not critical to the functioning of the invention. Before the clip 408 is slid over an edge of the blade (not shown) of a surgical retractor or other surgical or medical instrument, the clip 408 is formed so the gap 413 defines a distance which is less than the width of the retractor blade (not shown). In the embodiment (not shown) where the first end (not shown) and second end (not shown) actually contact each other, the width of the retractor blade is less important. In both embodiments, upon mounting the clip 408 onto a surgical instrument, the first 409 and second ends 411 of the clip 408 are forced apart so as to exert an amount of force sufficient to securely attach the clip 408 to the retractor blade (not shown) during a surgical procedure. Most retractor blades (not shown for this embodiment) have a thickness between 0.048 inches and 0.105 inches. The average pull force required to securely attach the clip 408 to a 0.05 gage retractor blade is approximately 1.67 pounds but may vary according to specific requirements between approximately 1.54 and 1.86 pounds. The average pull force required to securely attach the clip 408 to a 0.105 gage retractor blade is approximately 4.64 pounds but may vary according to specific requirements between approximately 4.40 and 4.60 pounds. The clip 408 and attached light conducting fibers 205 are thus adjustably movable according to the desires of the physician. It is also contemplated by and therefore within the scope of the invention to add a coating (not shown) or textured (not shown) or altered inner surface geometry (not shown) to the contacting surfaces of the clip (not shown), which would increase the holding ability of the clip without increasing the exerted force.

The clip 408 is preferably made of a medically and biologically compatible plastic such as Bayer Lustran ABS, made by Lanxess Engineering Plastics Corp. and can be extruded or injection molded. Alternative materials could also be used such as stainless steel or nitinol.

As shown in cross section in FIGS. 5A and 5B, the clip 408 is configured to be quickly attachable to a retractor blade (not shown) by means of a friction or compression fit. It should be mentioned that it is contemplated to attach the device 400 to any medical device, thus the invention is not limited to being used with surgical retractors only. Attached to the clip 408 is at least one light conducting fiber 205 which is in light communication with a light source (not shown for this embodiment, but similar to light source 202 as discussed above). As shown in FIG. 5B, at least one light conducting fiber 205 is attached to the pad 308 by means of a pressure sentisitve adhesive 305. It should be mentioned that although not shown, magnetic means of attachment are also contemplated by and therefore within the scope of the invention.

As shown in FIG. 2 a plurality of light conducting fibers within a bundle 204 can be encased in a sheath 207. The sheath 207 is made of 85 Shore PVC in a diameter of approximately 0.135 inches when encasing three light conducting fibers 205. Different numbers or diameters of light conducting fibers 205 will, of course, required a different sized sheath 207. A standard snap fit connector 203 terminates the light conducting fiber bundle 204 and is configured to plug into the light source 202. An advantage of using a clip 208 mounted system is that it can quickly and easily be moved on the medical device as individual procedures may require.

Use

Using the present invention first requires the physician and staff to prepare the patient for a medical procedure, typically surgery, followed by creating an incision proximal and convenient to the underlying area to be treated. Next, a clip 208, 408 having light conducting fibers 205 attached or pad 308, 608 having light conducting fibers 205 attached is attached to the retractor blade 206 or other surgical or medical instrument (not shown). Following this, the retractor blade 206 or other surgical or medical instrument (not shown) inserted into the incision to provide access for the physician to conduct the intended procedure. Next the retractor system is locked in place mechanically or held in place by technicians through the duration of the procedure. The light source 202 is turned on, which results in light energy flowing through the light conducting fibers 205 and exiting via the “open” end to provide illumination inside the incised area of the patient's body, shown as a leg 1000 for purposes of illustration but not intended to be limited to any particular anatomical region. Following completion of the procedure, the light source 202 is turned off, the retractor blade 206 or other surgical or medical instrument (not shown) is unlocked where necessary and removed or just removed and the incision closed using well known techniques.

What is claimed is:

1. A device for securing light conducting fibers to a surgical device, comprising:
a. a mounting platform attachable to the surgical device and
b. means for attaching least one light conducting fiber to the mounting platform.

2. The device of claim 1 wherein the mounting platform is a clip defining a first end and a second end, the first and second ends almost meeting each other in an unstressed configuration to form a gap which when spread apart exert a force toward each other.

3. The device of claim 2 wherein the clip exerts a pull force between approximately 1.54 pounds and 1.86 pounds against a 0.05 gage retractor blade.

4. The device of claim 2 wherein the clip defines a first major surface into which at least one retaining channel is shaped and sized to receive and secure the light conducting fiber.

5. The device of claim 4 wherein the retaining channel defines an arc formed through the first major surface.

6. The device of claim 5 wherein the arc is greater than 180 degrees.

7. The device of claim 2 wherein the clip defines a first major surface and the at least one light conducting fiber is attached to the first major surface by an adhesive.

8. The device of claim 1 wherein the platform is a pad defining a first major surface and a second major surface.

9. The device of claim 8 wherein pressure sensitive adhesive is attached to the second major surface to secure the pad to the surgical device.

10. The device of claim 9 wherein the pad defines a first major surface into which at least one retaining channel is shaped and sized to receive and secure the light conducting fiber.

11. The device of claim 9 wherein the at least one light conducting fiber is attached to the first major surface by an adhesive.

12. An illumination device, comprising:
a. a mounting platform attachable to a surgical device; and
b. at least one light conducting fiber attached to the platform.

13. The device of claim 12 wherein the platform is a clip defining a first end and a second end, the first and second ends almost meeting each other in an unstressed configuration to form a gap which when spread apart exert an amount of force toward each other.

14. The device of claim 13 wherein the first and second ends of the clip exert a pull force between approximately 1.54 pounds and 1.86 pounds against a 0.05 gage retractor blade.

15. The device of claim 13 wherein the clip defines a first major surface into which at least one retaining channel is shaped and sized to receive and secure the light conducting fiber.

16. The device of claim 15 wherein the retaining channel defines an arc formed through the first major surface.

17. The device of claim 16 wherein the arc is greater than 180 degrees.

18. The device of claim 13 wherein the clip defines a first major surface and the at least one light conducting fiber is attached to the first major surface by an adhesive.

19. The device of claim 12 wherein the platform is a pad defining a first major surface and a second major surface.

20. The device of claim 19 wherein pressure sensitive adhesive is attached to the second major surface to secure the pad to the surgical device.

21. The device of claim 19 wherein at least one retaining channel extends across at least part of and is formed into the first major surface.

22. The device of claim 21 wherein the retaining channel defines an arc formed through the first major surface.

23. The device of claim 22 wherein the arc is greater than 180 degrees.

24. The device of claim 20 wherein the at least one light conducting fiber is attached to the first major surface by an adhesive.

25. The device of claim 12 wherein the at least one light conducting fiber is in light communication with a light source.

26. An illumination device, comprising:
a. a mounting platform attachable to a surgical device;
b. at least one light conducting fiber attached to the mounting platform; and
c. a light source in light communication with the at least one light conducting fiber to conduct light energy through the at least one light conducting fiber.

27. The device of claim 26 wherein the mounting platform is a clip defining a first end and a second end, the first and second ends almost meeting each other in an unstressed configuration to form a gap which when spread apart from the unstressed configuration exert an amount of force toward each other.

28. The device of claim 27 wherein the clip exerts a pull force between approximately 1.54 pounds and 1.86 pounds against a 0.05 gage retractor blade.

29. The device of claim 27 wherein the clip defines a first major surface into which at least one retaining channel is shaped and sized to receive and secure the light conducting fiber.

30. The device of claim 29 wherein the retaining channel defines an arc formed through the first major surface.

31. The device of claim 30 wherein the arc is greater than 180 degrees.

32. A method of using a surgical illumination device, comprising the steps of:
a. providing a mounting platform attachable to a surgical device, the platform having at least one light conducting fiber attached to it;
b. attaching the mounting platform to a surgical device;
c. connecting the at least one light conducting fiber to a light source;
d. locating the surgical device, attached mounting platform and at least one light conducting fiber at a patient's medical treatment site; and
e. energizing the light source, thereby illuminating the medical treatment site.
